(19) United States
(12) Patent Application Publication
(10) Pub. No.: US 2026/0174492 A1
(43) Pub. Date: Jun. 25, 2026

(54) ELECTROSURGICAL GENERATOR CONTROL SYSTEM

(71) Applicant: Applied Medical Resources Corporation, Rancho Santa Margarita, CA (US)

(72) Inventors: Michael Nguyen, Rancho Santa Margarita, CA (US); Igor Gorin, Costa Mesa, CA (US); Joanna Pang, Santa Ana, CA (US)

(21) Appl. No.: 19/541,794

(22) Filed: Feb. 17, 2026

Related U.S. Application Data
(63) Continuation of application No. 18/408,388, filed on Jan. 9, 2024, now Pat. No. 12,551,262.
Application No. 18/408,388 is a continuation of application No. 16/562,362, filed on Sep. 5, 2019, now Pat. No. 11,864,812.
(60) Provisional application No. 62/727,195, filed on Sep. 5, 2018.

(51) Int. Cl. A61B 18/12; A61B 18/00; A61B 18/14

(52) U.S. Cl. CPC A61B 18/1206; A61B 2018/00601; A61B 2018/0063; A61B 2018/00642; A61B 2018/00726; A61B 2018/00767; A61B 2018/00779; A61B 2018/00827; A61B 2018/00875; A61B 2018/00892; A61B 18/1445; H03F 2200/129; H03F 2200/451; H03G 2201/103; H03G 2201/307

(57) ABSTRACT

Systems and methods for enhancing surgical outcomes by providing generators having optimal RF output for sealing, fusing and/or cutting tissue or vessels under all dynamic conditions are described. Examples of dynamic conditions may include varying tissue impedance load due to electrosurgical operations or tissue affects, any operational conditions and commands determined by the surgeon, surgical procedure and/or device script. This is achieved by implementing a digital closed-loop control system within the electrosurgical generator to regulate voltage, current, and power of the RF output. The digital closed-loop control system may include an RF amplifier for generating RF energy, a feedback system for constantly monitoring the electrical characteristics, e.g., voltage, current, and power, of the supplied RF energy to a connectable electrosurgical instrument and a microcontroller for processing measurement data from the feedback system and adjusting the output of the RF amplifier to meet a desired regulation target under any varying conditions.

### CROSS-REFERENCE TO RELATED APPLICATIONS

This application is a continuation of U.S. patent application Ser. No. 18/408,388 filed Jan. 9, 2024 which application is a continuation of U.S. patent application Ser. No. 16/562,362 filed Sep. 5, 2019, now issue U.S. Pat. No. 11,864,812, which claims the benefit of and is a non-provisional of co-pending U.S. Provisional Application Ser. No. 62/727,195 filed on Sep. 5, 2018, which is hereby expressly incorporated by reference in its entirety for all purposes.

### TECHNICAL FIELD

The present disclosure is generally directed to electrosurgical generator systems and methods and more particularly to electrosurgical control systems configured for regulating dynamically the generator's output to provide optimal radiofrequency (RF) energy for sealing, fusing and/or cutting tissues or vessels

### BACKGROUND

Electrosurgical hand devices or instruments have become available that use radiofrequency (RF) energy to perform certain surgical tasks. Electrosurgical instruments may include one or more electrodes that are configured to be supplied with electrical energy from an electrosurgical generator. The electrical energy can be used to fuse, seal, or cut tissue to which it is applied. Examples of such electrosurgical or surgical instruments may include graspers, scissors, tweezers, blades or needles.

Electrosurgical instruments typically fall within two classifications: monopolar and bipolar. In monopolar instruments, electrical energy is supplied to one or more electrodes on the instrument with high current density while a separate return electrode is electrically coupled to a patient and is often designed to minimize current density. Monopolar electrosurgical instruments can be useful in certain procedures, but can include a risk of certain types of patient injuries such as electrical burns often at least partially attributable to functioning of the return electrode. In bipolar electrosurgical instruments, one or more electrodes is electrically coupled to a source of electrical energy of a first polarity and one or more other electrodes is electrically coupled to a source of electrical energy of a second polarity opposite the first polarity. Bipolar electrosurgical instruments, which operate without separate return electrodes, can deliver electrical signals to a focused tissue area with reduced risks.

Even with the relatively focused surgical effects of bipolar electrosurgical instruments, however, surgical outcomes are often highly dependent on surgeon skill. Enhanced generators have been made to reduce this dependency.

### SUMMARY

In accordance with various embodiments, an electrosurgical system for sealing, fusing and/or cutting tissue is provided. The electrosurgical system may include an electrosurgical generator and an electrosurgical instrument or device. The electrosurgical generator, according to the embodiments of the present invention, may include a digital closed-loop control system that regulates the delivery of electrosurgical or radiofrequency (RF) energy, adjusts the RF energy and in various embodiments measures and monitors electrical properties, e.g., phase, current, voltage and power, of the supplied RF energy to the connectable electrosurgical instrument. In various embodiments, the digital control system enhances accuracy while ensuring stability in the measurements and regulation of the voltage, current and power of the RF output. This provides the optimal RF output for sealing, fusing and/or cutting tissue/vessels under dynamic conditions, such as for example, variable loads, procedural or operational conditions.

In accordance with one aspect of the present invention, a digital closed-loop control system for use with an electrosurgical generator that supplies electrosurgical RF energy to a surgical site is provided. The digital closed-loop control system may include a feedback system monitoring continually electrical properties of the supplied RF energy and generating digital RF signals relating thereto and a microcontroller configured with a variable gain factor to regulate and control an RF amplifier that generates the supplied RF energy across a plurality of RF regulation modes to provide optimal RF output for surgical procedures under any surgical, operational or procedural conditions.

In accordance with a second aspect of the present invention, a method for dynamically controlling an electrosurgical generator that supplies electrosurgical RF energy to a surgical site through an electrosurgical instrument is provided. The method includes the steps of retrieving desired RF setpoints or target values for a plurality of RF regulation modes and generating RF energy at the desired RF setpoints; measuring electrical characteristics of RF output via at least one channel from a feedback system and communicating real and imaginary components of measured data to a microcontroller. The microcontroller, after receiving the transmitted data, performs power calculations to obtain magnitudes of measured data and tissue impedance load for each of the plurality of RF regulation modes.

The method further includes the steps of generating an error signal across the plurality of RF regulation modes and selecting one regulation mode based on the calculated error values; calculating a variable gain factor for each of the plurality of regulation modes using specific algorithms and selecting one variable gain factor based on calculated error values; determining output control signals for Buck and H-Bridge circuitry of an RF amplifier of the electrosurgical generator; and controlling an amount of RF output of the electrosurgical generator in response to the output control signals to maintain a desired output value of the generator.

In accordance with a third aspect of the present invention, there is provided an electrosurgical system for performing surgical procedures. The electrosurgical system may include an electrosurgical generator adapted to supply RF energy to a surgical site and an electrosurgical instrument connected to the electrosurgical generator. The electrosurgical instrument having at least one active electrode adapted to apply electrosurgical RF energy to tissue at the surgical site. The electrosurgical generator may include a primary FPGA (fully programmable gate array) which is configured to cause: generating error signals across a plurality of RF regulation modes and selecting one regulation mode; computing a variable gain factor for the plurality of regulation modes and selecting one variable gain factor; generating an integral signal by integrating the selected error signal and multiplying the generated integral signal by the selected variable gain factor; and driving duty cycles for Buck and H-Bridge circuitry of the RF amplifier using respectively a predicted output voltage and the generated integral signal.

In accordance with a fourth aspect of the present invention, an electrosurgical generator is provided. The electrosurgical generator may include an RF amplifier for supplying RF energy, a feedback system adapted to continually monitor electrical properties of supplied RF energy to generate digital RF signals relating thereto and a primary microcontroller programmed to compute a variable gain factor and a preload function that allows for dynamically controlling the supplied RF energy across a plurality of RF regulation modes and a plurality of RF resolution settings under any surgical, operational or procedural conditions.

In accordance with a fifth aspect of the present invention, a method for impedance evaluation of an electrosurgical instrument, connected to an electrosurgical generator, prior to performing surgical procedures is provided. The method includes the steps of initiating a low voltage mode or passive mode upon activation of the connected electrosurgical instrument; generating RF output limited to values defined by the low voltage mode; measuring electrical characteristics of the RF output and transmitting digitally the measured data to a microcontroller of the electrosurgical generator; calculating other electrical characteristics of the RF output based on the received measured data and transmitting the calculated results to a primary processor within the microcontroller; and determining whether the calculated results has met a certain criteria set by a device script of the connected electrosurgical instrument.

In accordance with a sixth aspect of the present invention, there is provided an electrosurgical generator that includes an RF amplifier for supplying RF energy and a microcontroller configured to dynamically control the supplied RF energy across at least one regulation mode from a plurality of RF regulation modes and a plurality of RF resolution settings.

In accordance with a seventh aspect of the present invention, there is provided an electrosurgical generator that includes an RF amplifier for supplying RF energy and a microcontroller configured to determine at least one of a variable gain factor and a preload function to dynamically control the supplied RF energy.

Many of the attendant features of the present inventions will be more readily appreciated as the same becomes better understood by reference to the foregoing and following description and considered in connection with the accompanying drawings.

### BRIEF DESCRIPTION OF THE DRAWING

The present disclosure is described in conjunction with the appended figures:

FIG. 1 is a perspective view of an electrosurgical generator in accordance with various embodiments of the present invention.

FIG. 2 is a perspective view of an electrosurgical hand device in accordance with various embodiments of the present invention.

FIG. 3 is a perspective view of an alternative embodiment of an electrosurgical hand device in accordance with various embodiments of the present invention.

FIG. 4 depicts a block diagram of an electrosurgical generator in accordance with various embodiments of the present invention.

FIG. 5 depicts, in greater detail, a block diagram of an embodiment of a feedback system within a control system of an electrosurgical generator.

FIG. 6 depicts, in greater detail, a block diagram of an embodiment of a primary microcontroller within a control system of an electrosurgical generator.

FIGS. 7-8 is a schematic illustration of operational modes and functional blocks of various circuitry and systems within a primary microcontroller of an electrosurgical control system of the present invention.

FIG. 9 depicts a block diagram of an embodiment a control system of an electrosurgical generator operating in a passive regulation mode.

FIG. 10 illustrates a flow diagram of an embodiment of a passive regulation mode operations or process of an electrosurgical generator according to the embodiments of the present invention.

In the appended figures, similar components and/or features may have the same reference label. Where the reference label is used in the specification, the description is applicable to any one of the similar components having the same reference label.

### DETAILED DESCRIPTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiments of the disclosure. It should be understood that various changes may be made in the function and arrangement of elements without departing from the spirit and scope of the invention as set forth in the appended claims.

This disclosure relates in general to electrosurgical systems. It specifically relates to a new generation of electrosurgical generators capable of regulating voltage, current and power of the RF output under dynamically changing impedance loads and control conditions.

Embodiments of the present invention are directed to systems and methods for enhancing surgical outcomes by providing generators having optimal RF output for sealing, fusing and/or cutting tissue or vessels under all dynamic conditions such as, for example, varying tissue impedance load due to electrosurgical operations or tissue affects and any operational conditions and commands determined by the surgeon, surgical procedure and/or device script. This is achieved by implementing a digital closed-loop control system to regulate voltage, current, and power of the RF output. The digital closed-loop control system may include an RF amplifier for generating RF energy, a feedback system for constantly measuring and monitoring the electrical characteristics, e.g., voltage, current, and power, of the supplied RF energy to a connectable electrosurgical instrument and a microcontroller for processing measurement data from the feedback system and adjusting the output of the RF amplifier to meet a desired regulation target under any varying conditions.

According to the embodiments of the present invention, the feedback system measures, via at least one channel, analog RF output and digitizes the measurements. The feedback system in various embodiments collects its voltage and current measurements simultaneously from the RF amplifier and digitizes the measurements through analog to digital converters (ADC). The feedback system is configured to process the digitized values, to derive real and imaginary components of the voltage and current RF output, and to supply the real and imaginary components to the primary microcontroller.

In accordance with the embodiments of the present invention, the primary microcontroller, calculates individual error values for voltage, current and power and based on the individual error values selects a regulation mode. The primary microcontroller in various embodiments calculates, using specific algorithms, a specific variable gain factor for each regulation mode that allows the electrosurgical system according to the embodiments of the present invention to have a critically damped step response under any variable conditions, e.g., surgical, operational or procedural.

In the following, the electrosurgical system and method according to the present invention is explained in detail with sections individually describing: the electrosurgical generator, the electrosurgical instrument and the digital closed-loop control system and method used according to the embodiments of the present invention for providing optimal RF output under any dynamically outside changing conditions.

In accordance with various embodiments, an electrosurgical generator is provided that controls the delivery of electrosurgical or radiofrequency (RF) energy, adjusts the RF energy and in various embodiments measures and monitors electrical properties, e.g., phase, current, voltage and power, of the supplied RF energy to a connectable electrosurgical instrument to ensure optimal sealing, fusing and/or cutting of tissues or vessels. In various embodiments, the generator may include a feedback system that determines such electrical properties and through a microcontroller regulates and/or controls an RF amplifier that generates the required RF energy to provide the optimal RF output for sealing, fusing and/or cutting tissue or vessels under dynamic conditions, such as for example, varying loads, procedural or operational conditions.

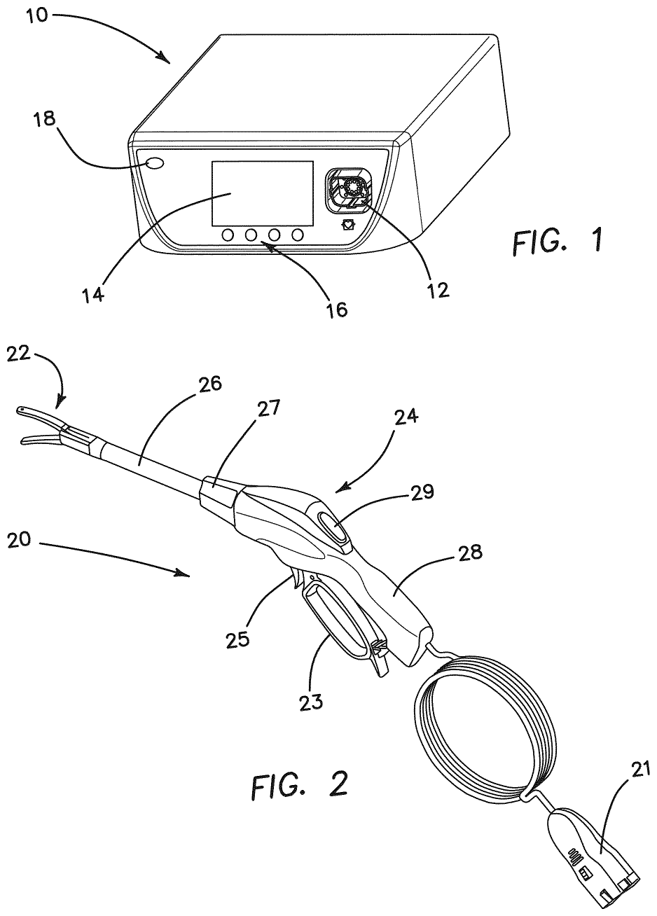

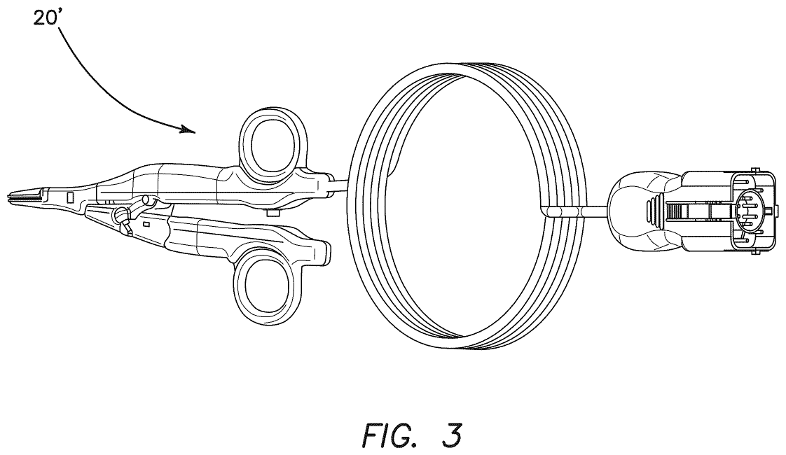

Referring first to FIGS. 1-2, an exemplary embodiment of an electrosurgical system for use in surgical procedure is illustrated. As shown in these figures, the electrosurgical system may include an electrosurgical generator 10 and a removably connectable electrosurgical tool or instrument 20. The electrosurgical hand device or instrument 20 can be electrically coupled to the generator 10 via a cabled connection with a device key or connector 21 extending from the instrument 20 to a device connector or access port 12 on the generator 10. The electrosurgical instrument 20 may include audio, tactile and/or visual indicators to apprise a user of a particular or predetermined status of the instrument 20 such as, for example, a start and/or end of a fusion operation. In some embodiments, a manual controller such as a hand or foot switch can be connectable to the generator 10 and/or instrument 20 to allow predetermined selective control of the instrument such as to commence a fusion operation.

In accordance with various embodiments, the electrosurgical generator 10 includes a display 14 that may indicate the status of the electrosurgical system including, among other information, the status of the one or more electrosurgical instruments and/or accessories, connectors or connections thereto, the state or operations of the generator and error indicators. The electrosurgical generator 10 in accordance with various embodiments of the present invention may include a user interface such as, for example, a plurality of buttons 16. The plurality of buttons 16 allows for user interaction with the electrosurgical generator 10. This user interaction may include, for example, requesting an increase or decrease in the electrical energy supplied to one or more instruments 20 that are coupled to the electrosurgical generator 10. In various embodiments, the generator 10 further includes a user-accessible power-on switch or button 18 that when activated powers the generator 10 and activates or initiates a self-verification system test of the generator. In other embodiments, the display 14 can be a touch screen display thus integrating data display and user interface functionalities.

In various embodiments, the electrosurgical generator 10 of the present invention is configured to output radiofrequency (RF) energy through the connectable electrosurgical instrument or hand device 20 to seal, fuse and/or cut tissue or vessels via one or more electrodes. The electrosurgical generator 10, according to the embodiments of the present invention, is configured to generate up to 300V, 8 A, and 375 VA of RF energy and it is also configured to determine a phase angle or difference between RF output voltage and RF output current of the generator during activation or supply of RF energy. In this way, the electrosurgical generator 10 regulates voltage, current and/or power and monitors RF energy output (e.g., voltage, current, power and/or phase). In one embodiment, the generator 10 may stop, terminate or otherwise disrupt RF energy output under predetermined conditions. By way of example, these predetermined conditions may be any of the following conditions: when a device switch is de-asserted (e.g., fuse button released), a time value is met, and/or active phase angle and/or change of phase is greater than or equal to a phase and/or change of phase stop value indicating end of an operation such as fusion or cutting of tissue.

The electrosurgical instrument 20, according to the embodiments of the present invention, may include an elongate shaft 26 having a proximal end coupled to or from which an actuator 24 extends and a distal end coupled to or from which jaws 22 extend. A longitudinal axis extending from the proximal end to the distal end of the elongate shaft 26. In one embodiment, the actuator 24 may include a movable handle 23 which is pivotably coupled to a stationary handle or housing 28. The movable handle 23 is coupled to the stationary handle or housing 28 through a central or main floating pivot. In operation, the movable handle 23 is manipulated by a user, e.g., a surgeon, to actuate the jaws 22 at the distal end of the elongate shaft 26, and thereby, selectively opening and closing the jaws 22. When tissue or vessels are grasped between the jaws 22, a switch or button 29 is activated by the surgeon to seal, fuse and/or cut the tissue/vessels between the jaws 22. Once the button 29 is activated, associated circuitry or contacts are connected to connect appropriate electrodes of the jaws with associated connections of the generator 10 to supply RF energy to tissue grasped between the jaws 22 or otherwise in contact with the one or more electrodes of the jaws.

In various embodiments, the electrosurgical instrument 20 further includes a mechanical or electrical cutting blade that can be coupled to a blade actuator such as a blade lever or trigger 25 of the stationary handle or housing 28. The cutting blade is actuated by the blade trigger 25 to divide or cut the tissue between the jaws 22. In various embodiments, a blade slider is connected to the blade trigger 25 and a protrusion extends from a proximal portion of the blade slider into an opening in one end of the blade trigger connecting the components together. The other end of the blade trigger is exposed and accessible by the user with the blade trigger 25 being pivotable about a trigger pivot at or near the mid-point of the blade trigger. As such, as the blade trigger 25 is pulled or rotated by the user proximally, the end of the blade trigger connected to the blade slider slides or moves the blade slider distally. Integrated with or attached to a distal end of the blade slider is a cutting blade, knife or cutting edge or surface. As such, as the blade slider translates longitudinally through a blade channel in the jaws, tissue grasped between the jaws 22 is cut. In one embodiment, the cutting edge or surface is angled to facilitate cutting of the tissue between the jaws 22. In various embodiments, the cutting blade is a curved blade, a hook, a knife, or other cutting element that is sized and configured to cut tissue between the jaws 22.

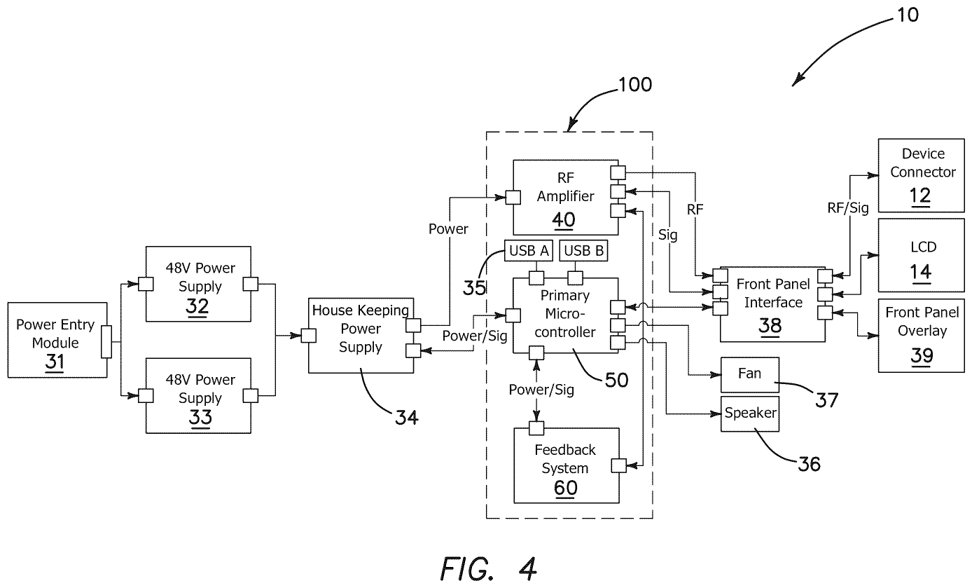

In accordance with various embodiments, the elongate shaft 26 comprises an actuation tube or rod coupling the jaws 22 with the actuator. In one embodiment, the actuator includes a rotation shaft assembly including a rotation knob 27 which is disposed on an outer cover tube of the elongate shaft 26. The rotation knob 27 allows a surgeon to rotate the shaft of the device while gripping the actuator. In various embodiments, the elongate shaft 26 is rotatable 360 degrees and in other embodiments, rotation of the elongate shaft 26 is limited to 180 degrees, i.e., ninety degrees clockwise and ninety degrees counter clockwise. FIG. 3 illustrates an alternative embodiment of an electrosurgical hand device 20′ connectable to the electrosurgical generator 10. The electrosurgical hand device 20′ is similar but includes different features and has a different surgical use than the electrosurgical hand device 20.

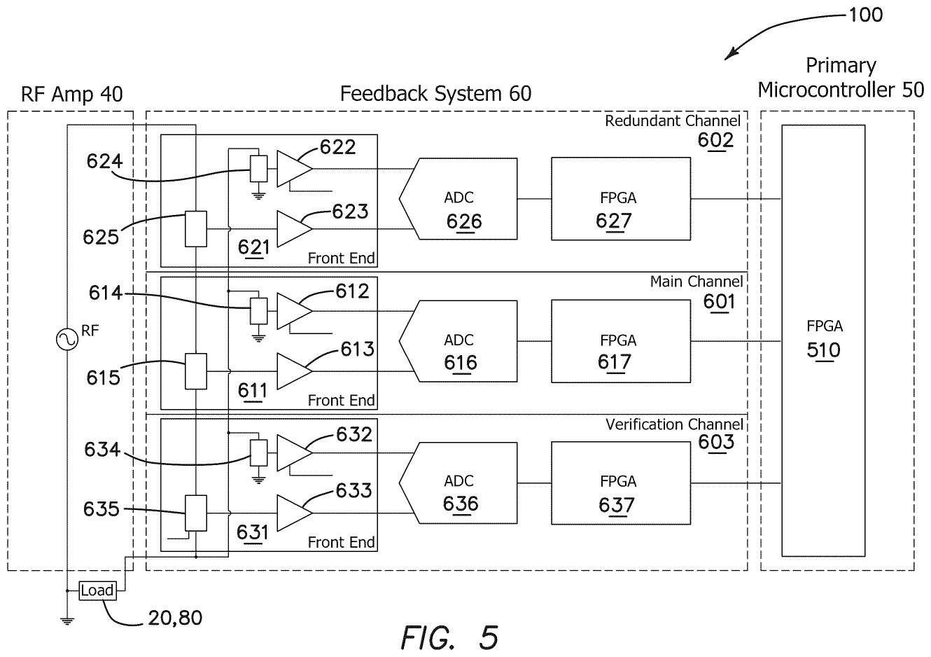

Referring next to FIG. 4, a block diagram of an electrosurgical generator 10 according to the embodiments of the present invention is shown. As shown in this figure, the electrosurgical generator 10 may include a power entry module 31, e.g., an AC main input, coupled to a power supply module, e.g., two 48V DC power supplies 32, 33. The power supply module converts the AC voltage from the AC main input to a DC voltage and via a house keeping power supply 34 provides power to various circuitry of the generator 10 and in particular supplies power to an RF amplifier 40 that generates or outputs the RF energy. In one embodiment, the RF amplifier 40 may include a Buck and H-Bridge circuitry to convert a DC voltage input into an RF output and in another embodiment into a variable amplitude 350 kHz sine wave. The DC voltage input is a 96V DC input that is generated by the two 48V DC power supplies 32, 33 coupled in series. One of the 48V DC power supply 32, 33 is configured to generate low voltage rails and in particular supply standby voltage to power on the generator 10.

According to the embodiments of the present invention, the electrosurgical generator 10 further includes a control system or a digital integral servo control system 100 to regulate and control the RF output. As shown in FIG. 4, the control system 100 may include the RF Amplifier 40, a primary microcontroller 50 and a feedback system 60. The RF output and in various embodiments the amplitude of the RF waveform output is controlled and regulated by the electrosurgical control system 100 which is embedded or integrated within the electrosurgical generator 10. The control system 100 varies between regulating voltage, current, or power of the RF output generated by the RF Amplifier 40. In various embodiments, the feedback system 60 measures the RF output and, after processing the measured data, digitally feeds the RF output's real and imaginary components to the primary microcontroller 50. The primary microcontroller 50, according to the embodiments of the present invention, processes the received data from the feedback system 60 and adjusts the output of the RF amplifier 40 to meet a desired regulation target. In various embodiments, the feedback system 60 comprises of analog input, digital processing and digital output.

In various embodiments, the electrosurgical generator 10 logs all RF output data onto an internal memory device, e.g., a secure digital (SD) or non-volatile memory card. The memory device is configured to be read through an interface port 35, e.g., a universal serial bus (USB) port, on the electrosurgical generator 10. In various embodiments, the generator 10 is configured to copy the data from the internal memory device to a connectable portable storage device, e.g., a USB flash drive, through the interface port of the generator.

In accordance with various embodiments of the present invention, the electrosurgical generator 10 is further configured to provide RF output in three resolution settings or modes: low voltage, normal or medium voltage and high voltage ranges. In various embodiments, device scripts stored and located on connectable electrosurgical hand devices, e.g., instrument 20, and/or connectors coupled thereto, e.g., device key 21, are used to determine or set the RF output or voltage mode.

Reffering back to FIGS. 1 & 4 and in accordance with various embodiments, the electrosurgical generator 10 is configured to alert the surgeon when the vessel has reached a completed procedure state, e.g., a completed seal state, or if an error or fault condition has occurred. The electrosurgical generator 10 in various embodiments may include visual, tactile and/or audible outputs to provide such alerts or other indicators or information to the surgeon as dictated by the surgical procedure, device script or health or operational information regarding the device 20 and/or generator 10. In one embodiment, the generator 10 via a front panel interface 38 alerts the surgeon through the LCD display 14, which is integrated into a front panel of the generator, and in various embodiments provides specific audible alarm or informational tones through a speaker 36 also integrated into the front panel of the generator. The generator 10 in various embodiments may include a front panel overlay 39 that provides a user interface or access including navigational push buttons to allow user access to systems settings such as volume or display brightness. The front panel overlay 39 may also include the system power button or connection. In various embodiments, a fan system 37 is provided to assist in heat dissipation. Additionally, as illustrated in the FIG. 4, signal or sig represents connections that, for example, comprise of digital signals used to communicate information across systems and/or printed circuit boards, power represents connections that, for example, comprise of voltage rails used to power systems and/or printed circuit boards and RF represents connections that, for example, comprise of high voltage, high current RF energy used to seal, fuse or cut tissue or vessels.

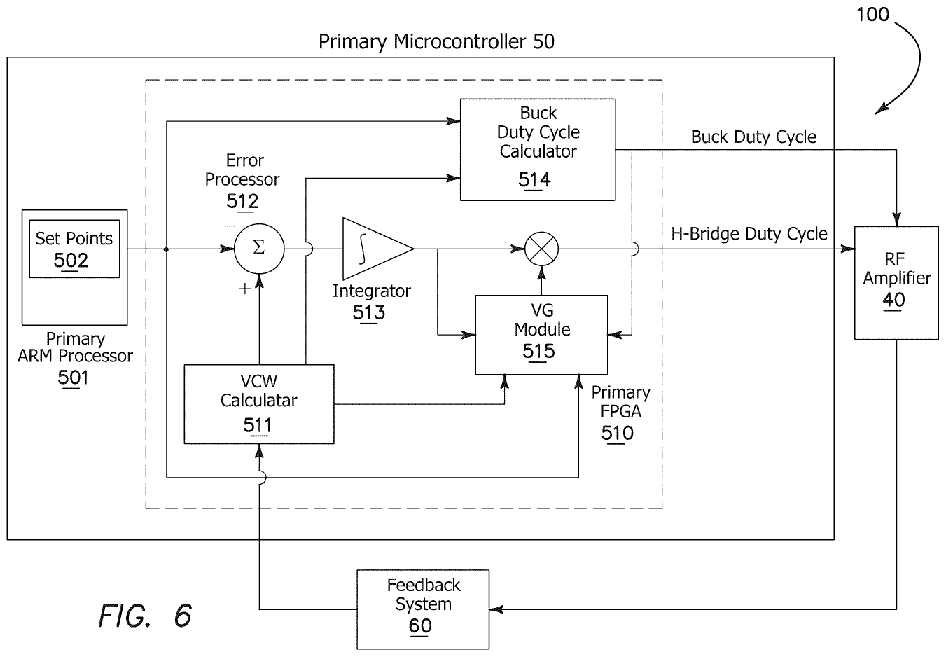

FIG. 5 illustrates, in greater detail, a block diagram of an embodiment of a feedback system 60 within the control system 100 of an electrosurgical generator 10. As described further above and also shown in FIG. 5, the control system 100 may include the RF Amplifier 40, the primary microcontroller 50 and the feedback system 60. In accordance with various embodiments of the present invention, the RF amplifier 40 generates an RF output and the feedback system 60 measures various electrical properties of the RF signal outputted from RF amplifier 40. According to the embodiments of the present invention, the verification system 60 may include a main channel 601, a redundant channel 602 and a verification channel 603. The main channel 601 and redundant channel 602 in various embodiments may include separate but identical components. Additionally, the main and redundant channels 601 and 602 follow separate but identical electrical paths and in one embodiment are both connected to the RF amplifier 40 and the RF output.

Similarly, components of the verification channel 603 are separate from the main and redundant channels 601 and 602 but are similar. In one embodiment, the verification channel 603 may include the same components as the main and redundant channels 601 and 602, but the components in the verification channel 603 have higher ratings, e.g., higher resolution and/or lower drift, and are often more costly. In another embodiment, the verification channel 603 may include the same components as the main and redundant channels 601 and 602. The verification channel 603 also follows a separate but identical electrical path as the main and redundant channels 601 and 602 and in one embodiment is connected to the RF amplifier 40 and the RF output. In various embodiments, the feedback system 60 measures analog RF output and digitizes the measurements. The feedback system 60 is configured to measure and digitize the RF output via at least one channel, e.g., main channel 601. In this embodiment, the feedback system 60 through the main channel 601 measures the analog RF output via a front end circuitry 611.

As shown in FIG. 5, the front end circuitry 611 may include a shunt resistor 615 coupled to a pre-amplifier 613 to measure the current of the RF output. In various embodiments, the front end circuitry 611 further includes a voltage divider 614 coupled to a pre-amplifier 612 to measure the voltage of the RF output. Outputs of the pre-amplifiers 612, 613 are supplied to an analog to digital converter (ADC) 616, thereby digitizing the current and voltage measurements. The digitized values are further processed to derive real and imaginary components of the voltage and current RF output. In various embodiments, the digitized values from the ADC 616 are supplied to a fully programmable gate array (FPGA) 617 of the feedback system 60. The FPGA 617 is configured for processing the digitized voltage and current measurements values to generate real and imaginary components of the voltage and current RF output using a discrete Fourier transform. The digital real and imaginary components are then supplied to the primary microcontroller 50 and, in one embodiment, via a serial communication protocol.

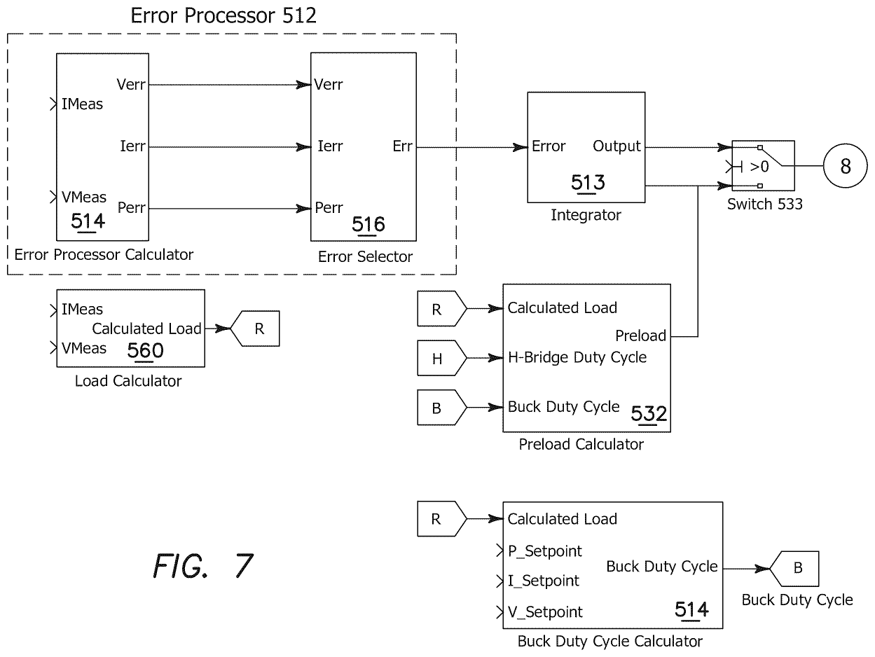

With reference to FIG. 6, a schematic illustration of an embodiment of a control system 100 depicting, in greater detail, a block diagram of an embodiment of a primary microcontroller 50 of an electrosurgical generator 10 is shown. As shown in this figure, the primary microcontroller 50 may include a primary ARM (advanced reduced instruction set machine) processor 501 and a primary FPGA (fully programmable gate array) 510. The primary ARM processor 501 is configured to establish desired output values, such as for example, voltage, current and/or power as setpoints 502. In various embodiments, the desired output values may be provided by a device script. In accordance with various embodiments, the primary FPGA 510 of the primary microcontroller 50 receives the digital real and imaginary components of the voltage and current measurements and calculates the magnitudes of the voltage, current and power of the RF output. The magnitude of the voltage, current and power of the RF output is calculated using a VCW (voltage, current, power) calculator 511, as shown in FIG. 6. Individual error values for voltage, current and power are also calculated by an error processor 512. In one embodiment, error values are calculated by subtracting a desired voltage, current and power setpoints from the measured magnitudes.

The error processor 512 calculates the relative error between the main channel measurements and the setpoints values 502, and based on the error values determines or selects a regulation mode. Accordingly, the error processor 512 determines which of the three regulation modes, e.g., voltage, current and power, should be reinforced or activated by the electrosurgical generator 10. In various embodiments, the calculated error values for the selected mode is integrated by an integrator 513 to generate an error signal that is directly proportional to and is used to correct the output of the RF amplifier 40.

According to the embodiments of the present invention, the calculated error values may also be used to determine a variable gain factor for each regulation modes, e.g., voltage, current and power, of the generator 10. The variable gain is configured to use a different predefined set of calculations or algorithm based on the selected regulation mode. As shown in FIG. 6, a VG (variable gain) module 515 is used to compute the variable gain value (Ki) for each regulation modes, e.g., voltage, current and power. The variable gain factor, according to the embodiments of the present invention, may be computed as a function of the voltage, current and power setpoints, the calculated outside impedance load or tissue load, the Buck voltage value as well as the value of the error integral or any combination thereof. As such, the variable gain in various embodiments provides critical step responses for all setpoints and impedance load conditions or any changes thereto. In other words, the variable gain according to the embodiments of the present invention allows for the electrosurgical generator 10 to be critically damped under any varying conditions such as, for example, surgical, operational and procedural conditions. In various embodiments, the variable gain factor may be recalculated on a predetermined schedule or timing such as, for example, every period of the RF output.

In accordance with various embodiments and with further reference to FIG. 6, the primary microcontroller 50 is configured to predict the necessary output voltage of the generator 10 to regulate the RF amplifier 40. In various embodiments, the primary FPGA 510 of the primary microcontroller 50 may use the calculated impedance loads and the voltage, current and power setpoints to predict the necessary voltage of the generator 10. The predicted value is then used by a Buck Duty Cycle calculator 514 to calculate a duty cycle value for a pulse width modulator (PWM) of an integrated Buck circuit of the RF amplifier 40. On the other hand, the product of the error integral and the calculated variable gain factor for the selected mode (Kᵢ*∫e(t)) may be used to derive a duty cycle value for an H-Bridge circuit of the RF amplifier 40. As such, the control system 100 according to the embodiments of the present invention is capable of providing dynamic regulation of the variable or varying RF output of the generator 10. In various embodiments, the electrosurgical generator 10 may be switching between voltage, current and power regulation modes. In such embodiments, the control system 100 is configured to perform a preload calculation or preload function, the details of which will be discussed further down below, to provide a gradual, non-disruptive transition in the RF output.

The control system 100, according to the embodiments of the present invention, provides regulation of RF output under dynamically changing impedance loads, e.g., due to electrosurgical operations or electrosurgical tissues affects, and control conditions, e.g., device scripts or user operations. The control system 100 being configured with a variable gain rather than a fixed gain allows the control system 100 to adjust for different load impedances and output voltages and thus not be limited to be optimized, e.g., for the lowest load impedance and/or highest output voltage. The control system 100 is also configured to account for the system becoming over damped as impedance increases that can result in non-optimal phase margin and dynamic or unpredictable behavior and thus affect the ability of the control system 100 to track or follow dynamic commands, e.g., device script operations. The control system 100 of the generator ensures that tissue electrosurgical effects, such as for example, sealing, fusing or cutting, are optimized through critical responses of the control system to dynamically changing tissue impedance conditions and operational conditions and commands determined by the surgeon, surgical procedure and/or device script.

As described further above, the feedback system 60 according to the embodiments of the present invention may include a second channel, e.g., the redundant channel 602, which is nearly identical to the main channel 601. The measurements from the redundant channel 602 and the resulting calculations are being constantly compared to the measurements and calculations of the main channel 601 to verify the operation of the main channel 601. As such, if the main and redundant channels 601 and 602 have differing measurements or calculations, then a generator error is recognized and the supply of RF energy halted.

In accordance with various embodiments, the feedback system 60 may include various other systems and circuitry, e.g., a sampler or other calculator (not shown in the figures), to provide sampling and/or other calculations as required by the electrosurgical control system 100 of the present invention. In various embodiments, the feedback system 60 measures analog voltage and current of the RF output of the RF amplifier 40 and in various embodiments the feedback system 60 takes a predetermined number of samples per each RF output cycle operating at 350 KHz for each measurement of voltage and current. In some embodiments, the feedback system 60 may utilize demodulations and transforms to obtain zero frequency components or filtering out unwanted higher order frequency harmonics out of the measured voltage and current values. As described further above, the feedback system 60 communicates or transmits, e.g., serially, the measured real and imaginary voltage and current values to the primary microcontroller 50.

In what follows, operational modes and functional blocks of various circuitry and systems within the primary FPGA 510 will be explained in detail with sections individually describing: the VCW calculator 511, the error processor 512, the integrator 513, the Buck Duty Cycle calculator 514 and the VG module 515.

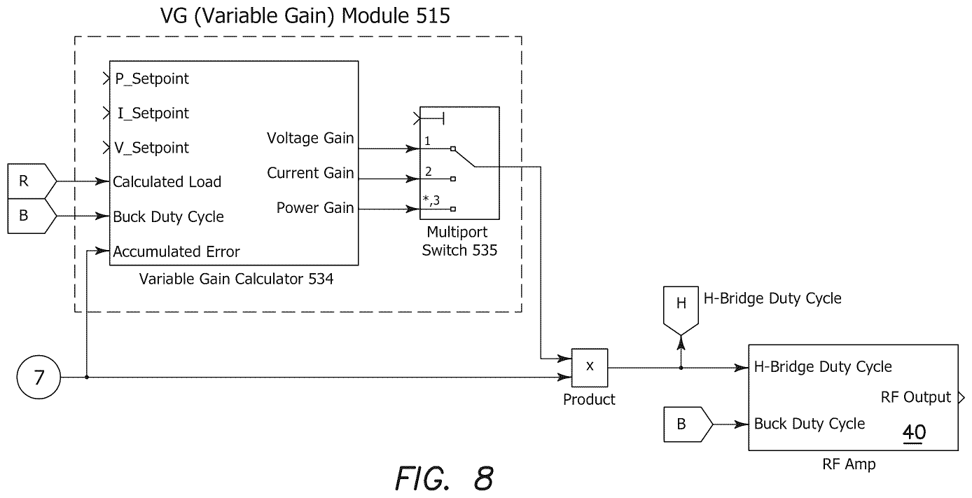

FIGS. 7-8 is a schematic illustration of operational modes and functional blocks of various circuitry and systems within a primary microcontroller 50 of an electrosurgical control system 100 of the present invention. According to the embodiments of the present invention, the primary FPGA 510 receives the measured real and imaginary voltage and current components or values from the feedback system 60 and uses these components to calculate their respective root means square (RMS) magnitudes using the VCW calculator 511. The VCW calculator 511 may further include a load calculator 560 (best shown in FIG. 7). The load calculator 560 uses the feedback system voltage and current measurement values to calculate the impedance load or tissue load. In some embodiments, filtered voltage and current measurement values are used for calculating the impedance load.

The primary FPGA 510 is further configured to perform error processing using the error processor 512. As shown in FIG. 7, the error processor 512 may include an error calculator 514 and an error selector 516. The error processor 512 calculates the error between the main channel measurements from the feedback system 60 and the setpoints values and determines which regulation mode is required for the correction of the RF output power. This is achieved by calculating the relative error between the setpoints and the measurements and in various embodiments this error calculation is performed simultaneously on voltage, current, and power by the error calculator 514. The error processor 512 utilizes the error selector 516 for determining which regulation mode needs to be enforced by the electrosurgical generator 10. Accordingly, the error selector 516 will select the regulation mode based on the most positive calculated error value. As such, the error with the most positive value will dictate which regulation mode is to be used by the electrosurgical generator 10. The primary FPGA 510 in various embodiments also normalizes the calculated magnitudes with respect to its maximum count value and then converted to floating point values.

The integrator 513 is constantly integrating the error with the most positive value, e.g., selected regulation mode. In operation, since the RF amplifier 40 may be switching between different RF regulation modes, e.g., voltage, current and power regulation modes, the integrator 513 needs to be preloaded with another value that allows the RF output to stay constant while transitioning between various regulation modes. For this purpose, a preload function or preload calculator 532 is implemented within the primary FPGA 510 (best shown in FIG. 7). The preload function or calculator 532 is configured to calculate the variable gain for the mode to which the RF amplifier is transitioning to and preload this value into the integrator 513 using a relay or switch 533 (best shown in FIG. 7). The preload function is calculated using the counts for the Buck and H-Bridge circuitry of the RF amplifier and the calculated tissue impedance load. This ensures a seamless transition between various regulation modes.

The primary FPGA 510 provides a variable integral control system to dictate the output for the Buck and H-Bridge (best shown in FIG. 8) controls of the RF amplifier 40. In various embodiments, variables used by the variable integral control system may include, for example, impedance load or tissue load calculations, setpoints for voltage current and power as well as the calculated RMS magnitude for the voltage, current, and power. The load calculator 560 may use filtered voltage and current measurement values for calculating the impedance or tissue load. In some embodiments, the variable integral control system only directly regulates voltage and in order to regulate current or power, a corresponding voltage value must be calculated. In various embodiments, the Buck duty cycle calculator 514 (best shown in FIG. 7) uses the calculated impedance load and the setpoints for voltage, current and power to predict where the output voltage of the RF amplifier 40 should be. The predicted voltage value is then used to generate the counts for the integrated Buck PWM circuit of the RF amplifier 40. The output voltage of the Buck PWM circuits of the RF Amplifier 40 sets the main voltage rails of the integrated H-Bridge PWM circuit of the RF amplifier 40.

According to the embodiments of the present invention, using the prediction set forth by the variable integral control system, the primary FPGA 510 sets counts for the Buck PWM circuit of the RF amplifier 40 and in various embodiments responds quickly to reach roughly close to the desired output value, e.g., the predicted voltage value. In various embodiments, the primary FPGA 510 drives PWM signals to the Buck and H-Bridge (best shown in FIG. 8) configurations or circuitry of the RF amplifier 40. In various embodiments, the determination of the PWM signals for the H-Bridge configurations is used to fine tune the RF output to the desired output. The duty cycle for the H-Bridge circuit of the RF amplifier 40 is defined by the multiplication of the calculated variable gain factor and an integral signal or error integral for the selected mode (best shown in FIG. 8). As can be seen in FIG. 8, the VG (variable gain) module 515 may include a variable gain calculator 534 and a multiport selector 535. The variable gain calculator 534 calculates the variable gain for each regulation mode, e.g., voltage, current and power, and selects the appropriate variable gain factor based on the same criteria that was used by the error processor 512, e.g. the error with the most positive value. The calculated variable gain may be defined as a function of the calculated impedance load, voltage, current and power setpoints, the Buck voltage value and the integral error or accumulated error. In various embodiments, the primary FPGA 510 converts respective numerical duty cycle counts to drive the PWM signals that controls the Buck and H-Bridge configurations.

In various embodiments, the primary ARM processor 501 verifies the validity of the setpoints and ensures the setpoints for voltage, current, and power meet the threshold for the mode the electrosurgical generator 10 is operating in. In accordance with various embodiments, calibration values are stored in an EEPROM of the feedback system 60. These values are specific predefined coefficients used to eliminate discrepancies or tolerances on the feedback system 60. In various embodiments, all three channels 601, 602 and 603 have calibration values for voltage, current, and power for normal or medium, high, and low voltage modes with the exception of the verification channel 603 not having a low voltage mode. The modes as such dictates the correct calibration coefficients for voltage, current, and power being used in the servo calculations. This also is based on the regulation mode the generator is operating in.

In various embodiments, the error processor 512 further includes one or more constants, such as a normalization factor, error coefficient and/or point positions (useful for floating point conversions). In various embodiment, the primary microcontroller 50 calculates the error between the main channel measurements and the setpoint values to determine which regulation mode to be used for the correction of the servo, e.g., the output of the RF energy. In various embodiments, the primary microcontroller 50 uses the calculated measurements and the error processor coefficient to obtain an absolute measurement. With this absolute measurement, the primary microcontroller 50 uses the calibration coefficient to obtain a calibrated absolute measurement and with the normalization factor obtains a relative measurement. The primary microcontroller compares the difference between the relative measurement and the setpoint established by the primary processor 501 to determine the relative error.

In accordance with various embodiments, the primary microcontroller 50 using multiplexers provide the respective values of the relative error to be calculated for voltage, current and power and comparisons are performed between the calculated errors to output the greatest or largest positive error to determine the regulation mode for the generator.

Using the selected regulation mode and its corresponding voltage value, the primary microcontroller 50 calculates the voltage output needed for optimal operation of the generator 10. In various embodiments, as the control system 100 adjusts the output voltage, current and power output targets are translated into their respective voltages at calculated loads. The regulation mode then decides which calculated output will be used in the control system 100.

In various embodiments, the control system 100 operates as a variable integral control loop. Variables are the voltage, current and power measurements, setpoints, and load calculations and the system operates at a predefined frequency, e.g., 350 KHz frequency, with the ability to switch between integral control loops. The electrosurgical generator 10 as such provides a control system for voltage, current and power driving sources and thus provides a generator integral control loops for current, voltage and power. Additionally, since switching between the integral control loops occurs when regulation modes are changed, the control system 100 implants the preload function for each mode, i.e., voltage, current and power, to ensure a smooth transition between the regulation modes.

In accordance with various embodiments, the feedback system 60 may include three channels: the main channel 601, the redundant channel 602 and verification channel 603. The main and redundant channels 601 and 602 are largely identical while the verification channel 603 has similar functionalities to the main and redundant channels 601 and 602, but has higher resolution, lower tolerance, and lower drift components.

In accordance with various embodiments, each of the channels 601, 602 and 603 of the feedback system 60 may include an analog portion that attenuates and amplifies the RF voltage/current measurement signals. In various embodiments, RF voltage signals are attenuated by a network of resistor dividers before being differentially amplified to drive the ADCs (616, 626, 636). In various embodiments, all three channels 601, 602 and 603 have different sets of amplifier gain resistors to measure different voltage modes, i.e., a normal voltage mode and a high voltage mode. In various embodiments, the normal voltage mode includes voltages less than or equal to 166V and in high voltage mode, voltages less than or equal to 322V. In accordance with various embodiments, the main and redundant channels 601 and 602 have an alternative set of resistor configuration to more accurately measure lower voltages and in various embodiments voltages less than or equal to 10V. The verification channel's resistor dividers in various embodiments contain much lower tolerance and lower drift resistors than that of the main and redundant channels 601 and 602.

In accordance with various embodiments, the RF current measurement signal is taken across a shunt resistor (615, 625, 635) from each channel of the verification system 60. All shunt resistors 615, 625, and 635 in various embodiments are in series, so each channel measures the same current signal. The main and redundant channels 601 and 602 in various embodiments have an alternative set of shunt resistors to more accurately measure lower currents, e.g., currents less than or equal to 100 mA. The verification channel 603 has shunt resistors that are lower tolerance and lower drift than that of the main and redundant channels 601 and 602.

In accordance with various embodiments, the measured signals after the amplifiers (612, 613; 622, 623; 632, 633) are passed through filters for ADC input filtering. The verification channel 603 has filter components with much lower tolerance and lower drift than that of the main and redundant channels 601 and 602. In various embodiments, the filter of the verification channel has a steeper rolloff and thus has a steeper attenuation of higher frequencies.

In accordance with various embodiments, data conversion components are independent between each of the three channels 601, 602 and 603. The ADCs (616, 626, 636) convert the analog voltage and current measurement signals to discrete samples that are processed by the respective channel's FPGAs (617, 627, 637). The verification channel's ADC 636 has more resolution, e.g., more bits, and has lower drift than that of the main and redundant channels 601 and 602. In various embodiments, the verification channel's ADC 636 also has a local generated reference voltage to accurately set the input range of the ADC 636.

In various embodiments, the feedback system's FPGAs (617, 627, 637) performs I/Q demodulation on the discrete voltage and current measurement samples to obtain real and imaginary samples. The measured values are passed through a discrete Fourier transform to obtain the DC component of the real and imaginary values for the voltage and current measurements. In various embodiments, the verification channel 603 contains a locally generated digital voltage rail to accurately power its FPGA's I/O pins.

In accordance with various embodiments, each channel of the feedback system 60 independently communicates its data to the primary microcontroller 50 through independent communication connections. In various embodiments, the verification channel's data is only used by a self-verification system or process at predefined time or schedule, e.g., at the start-up of the generator 10. During the self-verification process, the verification channel's data is compared with the main and redundant channel's data to verify the accuracy and functionalities of the main and redundant channels 601 and 602. In various embodiments, throughout RF related operations, the main channel's data is the only set of data used by the control system 100 and the redundant channel's data is constantly compared with the main channel's data to ensure the main channel 601 is operating within predefined parameters and/or tolerances.

According to the embodiments of the present invention, the servo control system 100 of the electrosurgical generator 10 may include the RF amplifier 40, the feedback system 60 and the primary microcontroller 50. The feedback system 60 creates a path for a closed-loop system between the RF amplifier 40 and the primary microcontroller 50. The feedback system 60 in various embodiments measures the voltage and current of the supplied RF signals and calculates the real and imaginary components of the measurements within one or more channels 601, 602 and 603. In one embodiment, only one channel is provided for the feedback system 60, the main channel 601. In another embodiment, two channels are provided, the main and redundant channels 601 and 602. In yet another embodiment, three channels are provided, the main channel 601, the redundant channel 602 and the verification channel 603. The calculated components within the one or more channels are transmitted or communicated to the primary microcontroller 50.

In accordance with various embodiments, the main and redundant channels 601 and 602 are copies of one another and are used by the primary microcontroller 50 to monitor the voltage and current of the RF output during operation of the electrosurgical generator 10. The verification channel 603 is similar to the other two channels 601 and 602, but includes components, for example, that are more drift resistant and/or uses ADCs with higher resolutions. This channel, in various embodiments, is used on startup of the generator, where self-verification of the generator is performed. The feedback system 60 in various embodiments collects its voltage and current measurements simultaneously from the RF amplifier 40. In various embodiments, the generated RF signal produces a voltage across one or more internal loads, e.g., load 80 (best shown in FIG. 5), disposed inside the RF amplifier 40 or a tissue load, e.g., electrosurgical hand device 20, 20′. The feedback system 60 in various embodiments collects current being delivered by using its own shunt resistors (615, 625, 635) and measures the voltage across them. To measure voltage, the feedback system 60 provides three voltage dividers (614, 624, 634) which are parallel to the load 20, 80. All measurements in various embodiments are converted to their real and imaginary components by the FPGAs 617, 627, and 637. The real and imaginary components are sent to the primary microcontroller 50 causing the feedback system 60 to act as a feedback device between the primary microcontroller 50 and the RF amplifier 40.

In accordance with various embodiments, the feedback system 60 measures the analog RF output via front end circuitry 611, 621, 631. Front end circuitry may include shunts 615, 625, 635 coupled to respective pre-amplifiers 613, 623, 633 to measure the current of the RF output. In various embodiments, the front end circuitry may also include voltage dividers 614, 624, 634 coupled to respective pre-amplifiers 612, 622, 632 to measure the voltage of the RF output. Outputs of the pre-amplifiers are supplied to respective analog to digital converters (ADCs) 616, 626, 636 thereby digitizing the current and voltage measurements. The digitized values are processed to derive real and imaginary components of the voltage and current RF output. In various embodiments, the digitized values from respective analog to digital converters (ADC) are supplied to FPGAs 617, 627, 637.

In various embodiments, the electrosurgical generator 10 is configured to provide RF output in a low voltage mode during a passive impedance evaluation which is automatically set by the generator 10. According to the embodiments of the present invention, the electrosurgical generator 10 is automatically set to the low voltage mode prior to execution of any device script. The device script in various embodiments represents a procedural walkthrough of a surgical operation that may include the application and termination of RF energy to the tissue. During a medium or normal voltage mode, the electrosurgical generator 10 according to the embodiments of the present invention is configured for having an output RF energy up to 150V or 8 A and is mainly used in tissue sealing. During a high voltage mode, the electrosurgical generator 10 according to the embodiments of the present invention is configured for having an output RF energy up to 300V or 4 A and is mainly used in tissue cutting. During the low voltage mode, the electrosurgical generator 10 according to the embodiments of the present invention is configured for having an output RF energy up to 10V and 100 mA and is mainly used in passive tissue impedance evaluations and measurements at a level that does not create a physiological response in tissue.

In accordance with various embodiments, specific device scripts are stored on specific electrosurgical hand devices 20, 20′ that are optimized for a specific surgical procedure to produce consistent electrosurgical sealing and/or cutting of tissue. In various embodiments, RF output parameters or settings are defined in the device scripts and used by the electrosurgical generator 10 to regulate or control the RF output for the specific surgical procedure and/or electrosurgical hand device 20, 20′. The device script and associated RF output parameters in various embodiments are retrieved or transferred to the generator 10 when the electrosurgical hand device 20, 20′ is connected to the generator 10. In one embodiment, the primary ARM processor 501 may retrieve the device script from a memory storage attached to or integrated into the device key 21 that connects the electrosurgical device 20, 20′ to the electrosurgical generator 10.

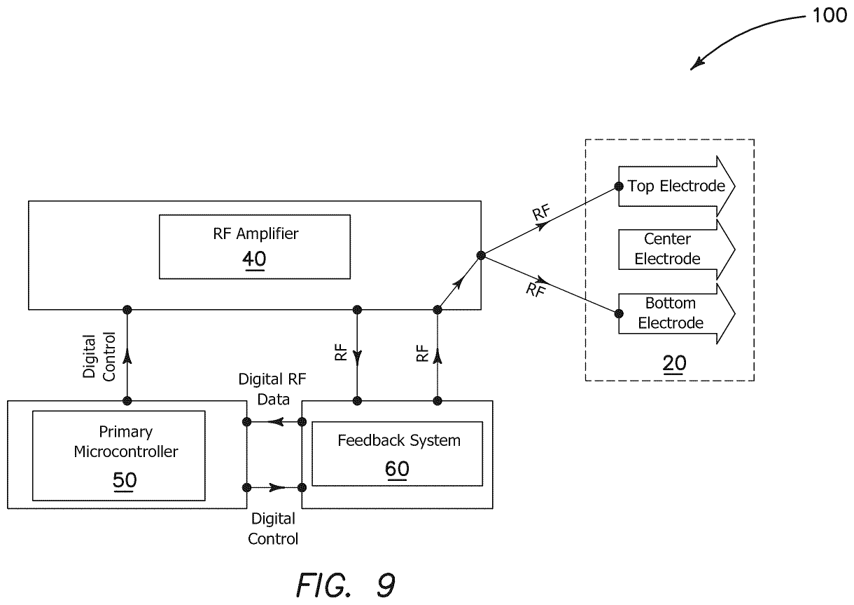

Referring next to FIG. 9, a block diagram of an embodiment a control system 100 of an electrosurgical generator 10 operating in a passive regulation mode is shown. In accordance with various embodiments of the present invention, the electrosurgical generator 10 is configured to provide a passive measurement regulation mode or low voltage mode to verify whether a connected electrosurgical hand device 20, 20′ can be used for specific surgical procedures such as, for example, sealing, fusing and/or cutting tissues or vessels. Thus, the passive regulation mode is triggered at a predetermined time, e.g., at each activation of the connected electrosurgical hand device 20, 20′. The passive mode is configured to detect open and/or short loads in the RF output path. In one embodiment, an open or short condition is predetermined and in various embodiments, is an acceptable impedance range or value defined by a device script included with the connected electrosurgical hand device 20, 20′ or otherwise associated with such electrosurgical hand devices 20, 20′. In various embodiments, the RF output for the passive mode has a lower static limit than other RF regulation modes and is used for a limited duration before normal RF regulation or operations of the electrosurgical generator 10 start. The low level RF output in various embodiments does not create a physiological response in tissue.

In various embodiments, when the electrosurgical generator 10 is operating in the passive mode, the RF amplifier 40 supplies a 350 KHz RF output via relays to the connected electrosurgical instrument 20, 20′. As described further above, the RF output in the low voltage mode or passive mode is limited to not more than 10V rms and/or not more than 100 mA rms. The control system 100 regulates and measures voltage and current via the feedback system 60. The primary microcontroller 50 determines if a short and/or open condition is encountered based on the device script and the measured voltage and current data from the control system 100. In various embodiments, one or more electrodes (best shown in FIG. 9) are used in passive mode and position or selection of the electrodes, e.g., top, center or bottom, may vary based on the connected electrosurgical device, e.g., device 20, 20′ and/or the position of the electrodes relative to the subject tissue or vessel.

In accordance with various embodiments, when a surgeon asserts a fuse or cut switch, the electrosurgical control system 100 initiates a passive impedance evaluation. The passive impedance evaluation triggers or identifies a fault, if a short or open condition is detected at the jaws 22 or distal working end of the electrosurgical hand device 20, 20′. If the passive impedance check is successful, the primary ARM processor 501 executes the full device script. In various embodiments, the primary ARM processor 501 instructs other circuitry of the electrosurgical generator 10 to output RF energy based on specific conditions, triggers, events and timing and according to specific settings. In various embodiments, the primary ARM processor 501 ensures the electrosurgical device is supplied specific RF energy according to specific output settings (voltage, current and power set points) and varies the RF output through the course of the procedure or surgical operation depending on various triggers defined by the device script.

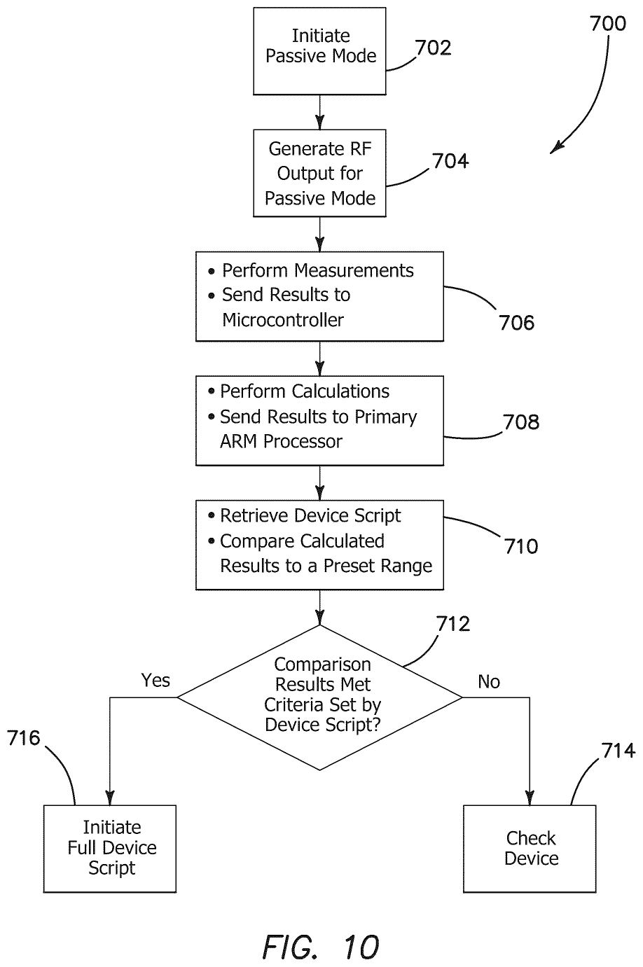

FIG. 10 illustrates a flow diagram of an embodiment of a passive regulation mode operations or process according to the embodiment of the present invention. The depicted portion of the process 700 begins in step 702 where the algorithm initiates the passive mode as a starting point. In accordance with various embodiments, the passive mode is initiated or triggered at each activation of the connected electrosurgical hand device 20, 20′ by a surgeon or other users. After initiating the passive mode, the processing goes to block 704 for generating RF output in the low voltage mode or passive mode and supplying RF energy to the connected electrosurgical hand device 20, 20′. In various embodiments, when the electrosurgical generator 10 operates in the passive mode or low voltage mode, the RF signal outputted from the RF amplifier 40 is limited to a specified voltage range (≤10V) and a specified current range (≤10 mA) for a range of 5-500 ohms resistance.

Once the RF output for the passive mode is generated, processing flows to block 706 where the feedback system 60 measures the electrical characteristics of the RF output. The control system 100, in accordance with various embodiments of the present invention, regulates the RF output to a set value as directed by the passive or low voltage mode and the feedback system 60 measures voltage, current, and/or phase from the main channel 601 and digitally feeds some or all of the measured values to the primary microcontroller 50. After completion of measurements and transmission of measured data, processing flows to block 708 where the primary FPGA 510 calculates or determines other electrical characteristics of the RF output based on the received data or readings and transmits some or all of the calculated results to the ARM processor 501 of the primary microcontroller 50. Other electrical characteristics of the RF output according to the embodiments of the present invention may include tissue impedance load and/or power. Once the calculated results are received by the primary ARM processor 510, the processing flows to block 710 where the primary ARM processor 501 retrieves the device script and compares the calculated results, e.g., calculated impedance load or tissue load, to a preset range set by the device script. In one embodiment, the device script is stored into a memory attached to or integrated into the device key or connector 21 that connects the electrosurgical hand device 20, 20′ to the electrosurgical generator 10.

A determination of whether the comparison results has met certain criteria set by the device script is made in step 712. Examples of the certain criteria may include, but not limited to, whether the comparison results or readings are within maximum and/or minimum values set by the device script. If the comparison results or readings are not between maximum and/or minimum values set by the device script, processing flows from block 712 to block 714 where an error is generated to notify the user or surgeon of an error and/or to check the electrosurgical device and/or its position relative to the tissue or vessel. In accordance with various embodiments, to supply RF energy after such a notification, the electrosurgical device 20, 20′ must be reactivated and the passive tissue impedance evaluation, e.g., passive mode or low voltage mode, be reinitiated.

If the comparison results or readings are between maximum and/or minimum values set by the device script, processing goes from block 712 to block 716 where the primary ARM processor 501 initiate the full device script to provide optimized RF energy for sealing, fusing and/or cutting tissue or vessel.

As described further above and in accordance with various embodiments, the control system 100 of the electrosurgical generator 10 may include one or more resolution settings and in various embodiments it includes three settings: low, normal or medium and high voltage setting. These resolution settings are different from the regulation modes and in some embodiments they require some adjustments to the circuitry that measures the RF output. Each setting is configured to require different hardware configurations for the feedback system 60 and/or different normalization algorithms in the calculations performed by the primary microcontroller 50. In various embodiments, the voltage measurement circuit of the feedback system 60 uses a different resistor selection or configuration for each of the three settings. In various embodiments, the current measurement circuit of the feedback system 60 uses the same resistor configuration for two of the settings, e.g., normal and high voltage settings, and a different resistor configuration for the low voltage setting.

In one embodiment, while the electrosurgical generator 10 is operating in the passive mode, the operations or process assigned to the primary ARM processor 501 may be performed via an FPGA. In other embodiments, other control systems may be incorporated therein. In yet another embodiment, a proportional, e.g., adjusting the system to reach setpoints, integral, e.g., measuring an area between error values and a time axis, prediction, e.g., predicting future errors based on a current error slope, architecture or any combination thereof may be included to supplement or replace the control system measurements, calculations and/or regulation.

In various embodiments, the electrosurgical generator 10 may supply an RF output having different waveform characteristics, e.g., square, providing non-sinusoidal periodic waveforms alternating between a minimum and maximum value; triangle, providing non-sinusoidal periodic waveforms with asymmetric ramps upward to a maximum value and downward to a minimum value; and/or sawtooth, providing non-sinusoidal waveforms with ramps upward to a maximum value and dropping sharply to a minimum value. In accordance with various embodiments of the present invention, the electrosurgical generator 10 may supply an RF output having different crest factor characteristics such as providing a ratio of peak value to effective value of a waveform, a peak amplitude divided by RMS value, and/or an ideal or perfect sine wave having a crest factor of 1.414.

The above description is provided to enable any person skilled in the art to make and use the electrosurgical devices or systems and perform the methods described herein and sets forth the best modes contemplated by the inventors of carrying out their inventions. Various modifications, however, will remain apparent to those skilled in the art. It is contemplated that these modifications are within the scope of the present disclosure. Different embodiments or aspects of such embodiments may be shown in various figures and described throughout the specification. However, it should be noted that although shown or described separately each embodiment and aspects thereof may be combined with one or more of the other embodiments and aspects thereof unless expressly stated otherwise. It is merely for easing readability of the specification that each combination is not expressly set forth.

Although the present invention has been described in certain specific aspects, many additional modifications and variations would be apparent to those skilled in the art. It is therefore to be understood that the present invention may be practiced otherwise than specifically described, including various changes in the size, shape and materials, without departing from the scope and spirit of the present invention. Thus, embodiments of the present invention should be considered in all respects as illustrative and not restrictive.

What is claimed is:

1. An electrosurgical generator comprising:
an RF amplifier for supplying RF energy; and
a microcontroller configured to dynamically control the supplied RF energy across at least one regulation mode from a plurality of RF regulation modes and a plurality of RF resolution settings.

2. The electrosurgical generator of claim 1 wherein the plurality of RF resolution settings comprises at least one of a low voltage setting, a medium voltage setting, and a high voltage setting.

3. The electrosurgical generator of claim 2 wherein the low voltage setting comprises an output RF energy up to 10V or 100 mA, the medium voltage setting comprises an RF output energy up to 150V or 8 A, and the high voltage setting comprises an output RF energy up to 300V or 4 A.

4. The electrosurgical generator of claim 1 wherein the at least one regulation mode comprises at least one of a voltage regulation mode, a current regulation mode, and a power regulation mode.

5. The electrosurgical generator of claim 1 wherein the at least one regulation mode is selected by an error processor having an error calculator and an error selector.

6. The electrosurgical generator of claim 5 wherein the error processor uses the error calculator to obtain individual error values for each of the plurality of RF regulation modes, with all individual error values being obtained simultaneously.

7. The electrosurgical generator of claim 6 wherein the individual error values are obtained by subtracting voltage, current and power setpoints from measured magnitudes of voltage, current and power.

8. The electrosurgical generator of claim 5 wherein the error processor uses the error selector to determine which regulation mode is to be enforced by the electrosurgical generator.

9. The electrosurgical generator of claim 6 wherein the error selector selects the at least one regulation mode based on a most positive error value.

10. The electrosurgical generator of claim 9 wherein the most positive error value passes through an integrator that includes a variable gain calculator.

11. The electrosurgical generator of claim 10 wherein the variable gain calculator is configured to calculate a variable gain factor using desired voltage, current, power setpoints, a measured tissue impedance load, measured magnitudes of voltage, current, and power of the supplied RF energy, and an output of the integrator.

12. The electrosurgical generator of claim 11 wherein the variable gain factor is calculated at each period of the supplied RF energy.

13. The electrosurgical generator of claim 10 wherein the integrator is continuously integrating the most positive error value.

14. The electrosurgical generator of claim 1 wherein the microcontroller is configured to predict an RF output of the electrosurgical generator using RF output setpoints and a calculated impedance load.

15. The electrosurgical generator of claim 14 wherein the predicted RF output value is used to derive a duty cycle for a Buck circuit of the RF amplifier.

16. The electrosurgical generator of claim 11 wherein the microcontroller uses the output of the integrator to derive a duty cycle for an H-Bridge circuit of the RF amplifier.

17. The electrosurgical generator of claim 16 wherein the microcontroller is further configured to calculate a preload function using a preload calculator to ensure a seamless transition between each of the plurality of RF regulation modes.

18. The electrosurgical generator of claim 17 wherein the preload calculator performs specific calculations to obtain the variable gain factor for the next regulation mode to which the electrosurgical generator is switching.

19. The electrosurgical generator of claim 17 wherein the preload function is calculated using the duty cycles of the Buck and H-Bridge circuitry of the RF amplifier and the measured tissue impedance load, or any combination thereof.

20. The electrosurgical generator of claim 17 wherein the preload calculator preloads the preload function into the integrator using a relay.
